(19) United States
(12) Patent Application Publication
(10) Pub. No.: US 2017/0106202 A1
(43) Pub. Date: Apr. 20, 2017

(54) FREQUENCY STIMULATOR

(71) Applicants: Urban Butinar, Polzela (SI); Dusan Petek, Celje (SI)

(72) Inventors: Urban Butinar, Polzela (SI); Dusan Petek, Celje (SI)

(21) Appl. No.: 15/105,916

(22) PCT Filed: Jun. 8, 2015
(86) PCT No.: PCT/IB2015/000886

§ 371 Date: Jun. 17, 2016

(30) Foreign Application Priority Data
Apr. 8, 2014 (SI) 201400134

(51) Int. Cl. A61N 2/00; A61M 21/02; A61N 2/02

(52) U.S. Cl. CPC A61N 2/006; A61N 2/02; A61N 2/002; A61M 21/02; A61M 2021/0044

(57) ABSTRACT

In some cases, a frequency stimulator that can control a dosage of impulses of various low frequencies of electromagnetic waves, uses a cell phone, a tablet, a laptop, and/or any other suitable devices that produce frequencies and can supply an inductive coil that is installed in a headband of the frequency stimulator in a way to produce a low frequency electromagnetic field with the basic purpose of achieving a positive response to the frequency of the human brain that responds to a programed frequency and adjusts to it, thus creating various states of consciousness in the human head. In some cases, the power source is connected to the headband in which there is the induction coil. In some cases, the height, shape and temporal lasting of the frequency impulses is controlled by software installed in a GSM or another source of power.

### BACKGROUND

Electromagnetic fields are omnipresent in our environment, yet they are invisible to human eye. People are daily surrounded by various sources of electromagnetic fields with various frequencies and strength. For example, the Earth's magnetic field turning the compass needle to the north, electricity lines, microwave ovens, televisions, computer terminals, hair driers, surveillance systems and cell phones.

Different researches have shown that impulse electromagnetic fields with little strength and extremely low frequency cause the raise of oxygen in blood (the speed of transition), help circulation and the cellular metabolism. The results are seen as better sleep, pain relief, reduction of inflammation, better energy level, bone healing, etc.

### BRIEF SUMMARY

The proposed invention is connected to a new product, which concept is enclosed in this patent application form. Some embodiments include a headband with an induction coil, which uses an application installed in a smart phone, tablet, lap-top, and/or any other programmable device that produces a suitable frequency. For the purpose of the text we will name this GSM (Global System for Mobile communications—original Groupe Spécial Mobile) as a synonym for all the devices which are capable of giving out frequency amplitudes as a result of using the energy of the GSM, which functions in such a case as a generator of low frequency impulses. In some embodiments, the headband has an induction coil installed, which functions as an emitter that emits the above mentioned low frequency impulses throughout the human body. In some cases, in the headband transmits the frequencies to the human head.

The subject of some embodiments of the invention is thus the following device: a frequency stimulator that is placed on the head. In this regard, some embodiments of the device influence the human body, more precisely the human head, to which regulated dosages of impulses on various frequencies of electromagnetic waves (EMW) are being released. These EMW have a positive effect on parts of the brain, hence causing various (depending on the strength and frequency of the EMW) positive impacts on the functioning of the brain, or gaining a new usage and focused working on the specific segments of the human brains in a way that their function is improved. Various inductive coils, depending on the number of turns, cross sections of the copper (or other suitable) wire and the surface of the iron (or other suitable) core, may use various currents to ensure the optimal frequency.

With some embodiments of the described frequency stimulator, the strength of created impulses is 0.6 mT, which is enough to affect brains and their functioning, but still far below the allowed limit of 100 mT, which is standard set for the safe usage of devices, and which still doesn't cause harmful consequences upon a human body. It is just strong enough for our body to detect the frequency and react to it by adjusting to the desired frequency, without suffering any harmful effects of the created electromagnetic impulses.

In the following sections of the patent application we will focus, as first in the world, on the source of EMW, such as the mobile telephone devices—GSM. It is the GSM devices, upgraded through the use of our software, will function as the generator of low frequency impulses that will be the source for the device (subject of this patent application) the headband with a coil to create the EMW at a frequency to achieve the positive effect on individual segments of the brains, which adopt to this frequency and create or have a positive effect on various states of conciseness of the human body.

With the innovative solution, which is the subject of some embodiments discussed in this patent application, we can turn out GSM device and the software for the android operating system (and any other suitable operating system) into a device that becomes a generator of low frequency impulses, which is usually stabilized with a crystal to ensure the precise and stable EMW frequency with normal temperatures for the most common use of the device (usually room temperatures). By doing this, some embodiments save users the purchase of an expensive low frequency impulse generator. Such generators already are available for purchase through the standard retail of electronic equipment in various frequency ranges and price ranges.

In some embodiments, the device (headband), on the other hand, is made from two parts. First part is the hoop that carries the structure. This hoop can be made of plastic, wood, hollow rubber, and/or any material similar to those mentioned, which can be elastic, biased, and/or resilient to a certain degree. The other part of the device is the induction coil that emits various EMW frequencies. In some embodiments, the induction coil is an electronic receiving-emitting device, that has the envelope with an iron (Fe) core and a copper (Cu) wire wrapped around it. The induction coil is an inductive emitter of various low frequency impulses EMW, which are regulated by applying impulse electric current of various strength to it, expressed by the unit of milliamps (mA). By supplying the selected current, some embodiments of the induction coil create a magnetic field of desired strength of EMP between 0.03 and 0.08 T. These values will be further described in the technical description part of our innovation that is the subject of this patent application.

Some embodiments provide a technological-constructional solution to the problem of the usage of existing materials accessible on the free market that are sold as materials or electronic elements, all of which achieve a higher value class after being treated as suggested with this patent application. With the disclosed systems and methods for treatment, some embodiments of the mentioned material and electronic devices are placed as a new product, which fulfils its technical and applicable function.

In some embodiments, the technical problem, to which some embodiments of the invention are the subject of this patent application, proposes how to create a headband that will have in it an inductive coil and that is configured to link the inductive coil within the headband with the GSM device by the use of a software solution. The software solution is not a subject of this patent application. With the help of this software solution it will be possible to determine the strength of the electric current for the purpose of the impulse production, which will create the EMW which will have a positive effect on individual fields—segments of the human body; in some embodiments, the human head.

The other important technical solution, which was already mentioned in the above text, is that some embodiments of the described systems and methods give a new functionality to the GSM devices, which are thus becoming the energy source to carry out the function previously carried out by the generators producing low frequency impulses.

If we want to better understand the main point of our patent application, we need to get to know how it works, or better said, we need to get familiar with the scientific discoveries on how EMW effects the human body, more specifically the relation between EMW and the human brain. The human brain often adapts itself, depending on the state of conciseness, to the needed strength of functioning—in this case the frequency of functioning. In some embodiments, the general medicine recognises that the closer a source of EMW is, the output of electromagnetic frequencies to the receiver of the radiation (in our case the human head), the stronger is the positive effect of such vibrations on a progressive scale, when considering the strength of it and the size of the repeating waves on places on a timeline. It is because of this that it is needed for the optimal functionality of our device, to have a source as close to the brain as possible. Hence, in some embodiments, the brain will be exposed to the positive effects of our EMW electromagnetic waves. For this purpose, in some embodiments, the induction coil is built into a headband, thus allowing us to reach the maximal coverage with the same strength of the EMW to the field of influence—said in other words, the parts of the brain we wish to expose to the dosage of electromagnetic waves.

The sensibility of the human organism, in this case the human head, for the electromagnetic field waves is connected to the frequency of the field, the strength of the field, the length of exposure to the positive radiation and the places of exposure. In some embodiments, the intensity of the EMW is expressed either as the strength of the electric field expressed in V/m (Volt per Meter) or as the magnetic field in T (Tesla). In the case of radiation from the GSM device at close range, the unit of W/cm² (Watt per square centimetre) is sometimes used. This is why some embodiments of the headband are connected with the GSM via a cable in such a way, that it is not close to any vital parts of the human body, thus preventing its undesired influence to other body parts.

On the other hand, it has been found that the low frequencies in the state of sleep, meditation and relaxation are always higher than in the state of raised functioning of brain activities such as concentration, learning or other forms of brain usage. The brain is a very sensitive apparatus, which is susceptible to outside influence and reoccurring impulses, which can be either audible, visual or in our case electromagnetic that triggers a reaction with which it changes the frequency at which the brain is functioning.

It has also been found that high frequency EMW have negative effects on various sensitive parts of the human body, as is the case with the central nerve system, the cardiovascular system, the hormones and the reproductive systems. Based on clinical and experimental finding, we can say that high frequency radiation of low energy (less than 10 .W/cm² or the frequency spectre from 0 to 300 GHz) are capable of changing the functional conditions of living organisms of various organizational structures (the impact factor is the temporal length of the radiation). On the other hand, it has been found that the low frequency EMW has a positive effect on various states of our brains, which is shown in the large number of various devices that have different effects, influence and heal various parts of the human body—in the case of the subject of this patent application, the head.

People have been using extremely low frequencies for changing the state of conciseness already in the distant past. Examples of this are meditations with the rhythmical sounds of drums, gong therapies, the constant flashing light of the fire that takes you into a trans-like state of relaxation. The monotonous sound of the wheels of the train while riding can also cause you to become sleepy. Various rhythms of music, with different beats per minute, can also have an effect on us. What is the difference between perceptions of Vivaldi's Four Seasons and the beats at the Rave party? In both cases the brains adjust to the outside impulses.

Some embodiments of the device that is the subject of this patent application—the frequency stimulator—are a product of knowledge gained throughout cultural backgrounds, contemporary researches, science and new technologies, with which we can mimic the functioning of the brain and stimulate it into a desired activity. In some embodiments, we do this with the help of short magnetic impulses, distributed in different rhythmic sequences on the temporal axis.

The basic functioning of some embodiments of our frequency stimulator is based on the usage of a personal cell phone, tablet, and/or other devices with a pre-installed application. In some embodiments, the GSM is used as a generator of above mentioned electric impulses, in combination with the inductive coil located in the headband that provides the necessary electromagnetic field of the desired frequency and speed of pulsation.

More precisely, in some embodiments, the electromagnetic inductive coil is placed into a polymeric headband that uses its own magnetic attributive flux (inductivity) to gain a positive effect on the head and the brain of a human, mostly upon the circulation of bodily fluids and the transition of electrolytes. With the right way of impulse stimuli of the coil and the right timing it causes different psycho-physical states of people as living beings.

In some embodiments, the inductive coils are connected with a cable with a GSM device which gives primary signals in desired frequency and time frame of the amplitude that the coil intensifies—strengthens and conveys to the body (especially the cranial lobe) by the means of an electromagnetic field. As the main component of the human body is H₂O, in some embodiments, the magnetic pulse applies itself as vibration of the tissue that affects the body parts we target through the primary function of the device (arousal).

As we indicated in the previous text, it is possible to use a telephone or tablet, or any other suitable programmable contemporary electronic device to generate the frequency. In some embodiments, one or more programs of desired frequencies are available as applications on Google, Android, another repository, and/or the iOS Apple store, however, they are not necessarily part of this patent application, but we will present those frequency areas and temporal impulses to the size we determined by several repeated tests and will exploit them for the purpose of our headbands.

We have determined that there are different conventional technical solutions, but have not found any with a combination of a GSM telephone with a software for emitting positive impulse frequency with the help of a single inductive coil functioning with technology of emitting the impulses of EMW—electromagnetic waves or frequencies to the human head.

There are several producers and suppliers of various types and price ranges, as well as purposes, strength and other categories of both generators of low frequency impulses and headbands, among which we will name just a few better known producers such as: Brain Sync from USA, Comptonic Devices Limited from Canada, IBVA Technologies Inc. from USA, The lucidity Institute USA, Microfirm, Inc. from USA, Mind Gear Inc. from USA or the device known under the brand of IMRS2000, which is the lead device in the field offering the holistic technical solution that in the sense of the innovative idea do come into the field of our device. The characteristic that is typically found with the mentioned devices and those produced by other producers is that they offer their own source of EMW impulses, meaning the power supply and the headbands use a lot of unnecessary electronic components and electronic systems that damper and filter frequencies and similar, or create specific voltage loops.

During the research of the innovative idea in the field of applied patents we have discovered there are tens of various patent applications that are usually connected to different kinds of generators of frequency impulses in a combination with various head sensors, electrodes, emitters etc., which are specified with different usages from healing specific pains in various body parts to psychiatric methods—therapies for healing mental problems of people.

The Slovenian patent SI 9500048—appears to teach a generator of a pulsating magnetic field proposes various frequencies from 2 to 24 Hz and a specific strength of the magnetic field (expressed in T) and the time impulses for healing various painful parts of the human body. In accordance with some embodiments, we have replaced this generator by a GSM device and also we do not use the electrodes as emitters of the magnetic field.

The capacity sensor of position SI 9800108—appears to use electrodes to determine the size of amplitude at an individual point. In some embodiments, our solution is based on the opposite principle, as we are sending specific saw-like amplitude determined as a temporal impulse, which grows from the determined basic frequency to the working or optimal size or strength of frequency and impulse.

The German inventor with the patent DE 1030485—appears to teach an appliance for carrying out magnetic therapy and the procedure for ensuring the current for magnetic therapy, which is all basically a device and an instrument for measuring the cardiovascular parameters and healing of such heart conditions. A closer examination of the electronic scheme of the circuitry shows the device appears to be based on the strength generator of frequency impulses, which is capable of receiving signals by the help of sensors. Hence it does not appear to overlap our innovative idea, although it really is a holistic strength based device for healing with the help of EMW we know as magnetic resonance.

The device for influencing biological processes, applied under EP 0995463, also appears to be a generator of a pulse field between 1 and 1000 Hz. This again means a strength based device, which has a wide spectre of uses in medicine by healing with EMW.

WO 1999/066986—appears to teach a device for healing with magnetic field consist of many magnetic coils, which use the EMW and the atom core spin resonance to set the diagnosis and consequently heal the specific illness. These appear to be resonating magnetic devices that use specific components of atom core and materials for healing and setting diagnosis of serious and cancer illnesses.

The solution that may appear to be most similar to ours is that of PCT/US 05/031867, when the inventor applied for patent for invention: That application appears to relate to a procedure and apparatus for modulating the effects of electromagnetisms on a human body. In this case, again, it appears that the EMW generator is a standalone unit, which is the source of various frequencies of electromagnetic waves and among other solutions suggests a headband with electronic components—but not with an inductive coil, which is one of the basic things in our patent application.

Some of the mentioned patented solutions and tens of those we did not mention, as well as many more that are not patented, use the technical solution of the usage of low frequency and high frequency devices that are based on the impulse electromagnetic waves, but may be significantly different from our invention for the following reason: in accordance with some embodiments, we are combining the usage of a cell phone, tablet, and/or other device that is discrete from the headband and that functions as a generator of electric impulses, which are emitted via conductor or wirelessly into a coil that forms the electromagnetic field. By changing the frequencies and a combination of various frequency areas, in some embodiments, we achieve a different effect on the brain in a way that we create the frequency of brain in their natural state—for example while sleeping, while relaxing, while prepared for fast learning or concentration. By processing specific frequencies we “tune” the brains into a desired state of consciences and working. We believe the following statement to be true until today nobody has connected a GSM, with the suitable software, with a headband receiver with inductive coil or in any other way combined these elements as they are presented in this application.

On the other hand, we have come to a conclusion and believe that our solution is unique and represents a novelty in the field, which is indicated in this patent application and its patent claims.

In accordance with some embodiments, the novelty is at least partially in the different design and the solution of technical problems, which were identified in the process of designing the device. The goal was set relatively high, but the main features of the procedure itself was a concept of a new way of usage of GSM devices that in the end allowed for the construction of the product that is presented (in at least some embodiments) by: a head earmuff and/or band for transition of low frequencies of electromagnetic waves with the help of the inductive coil, which, to our knowledge, has not yet been used in such a way in this technological branch. At the same time, we submit that this is a highly commercial product that has found a wide use, because of the unexpected results and useful characteristics and quality.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1: Depicts a cross-section of the basic concept of a headband that involves an inductive coil with a cable connection of the inductive coil to the source of power and emitter of pulsating frequencies—GSM, in accordance with a representative embodiment.

FIG. 2: Depicts a cross-section of the basic concept of our headband that involves the inductive coil with the wireless connection of the inductive coil to the source of power and emitter of pulsating frequencies—GSM, in accordance with a representative embodiment.

FIG. 3: Depicts a cross-section of the basic concept of our headband that involves multiple (two or more) inductive coils, in accordance with a representative embodiment.

FIG. 4: Depicts a cross-section of the basic concept of our headband that involves the inductive coil in a combination with acoustic earphones, in accordance with a representative embodiment.

FIG. 5: Depicts a cross-section of the basic concept of out headband that involves an inductive coil in a combination with a light source, in accordance with a representative embodiment.

### DETAILED DESCRIPTION

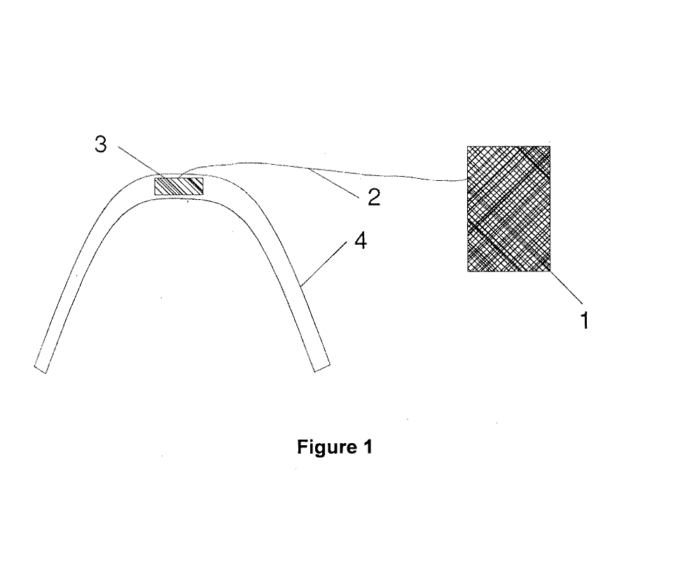

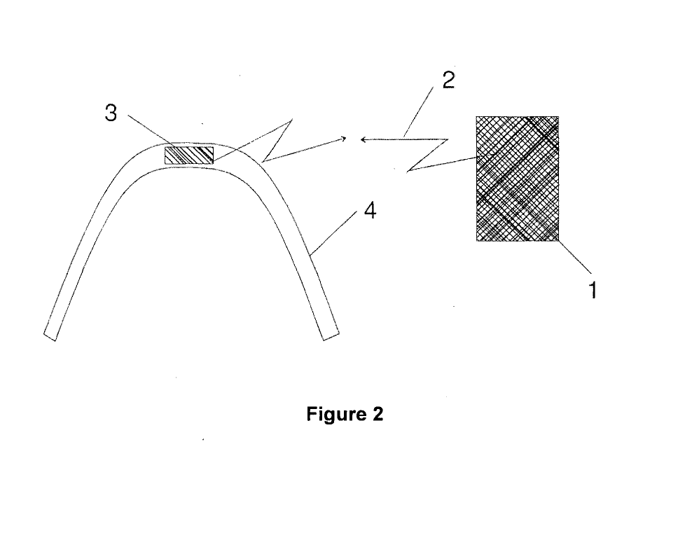

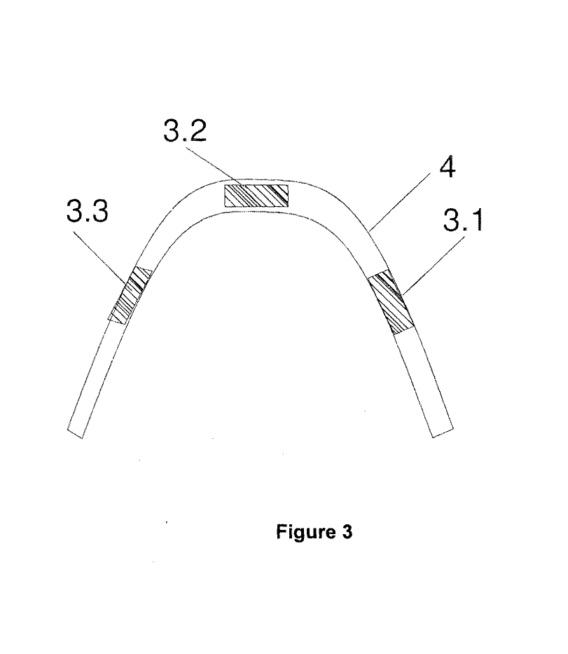

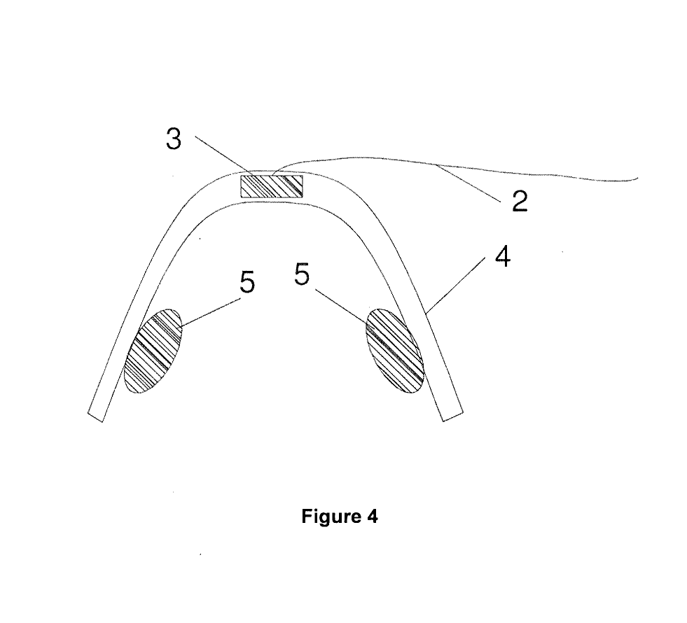

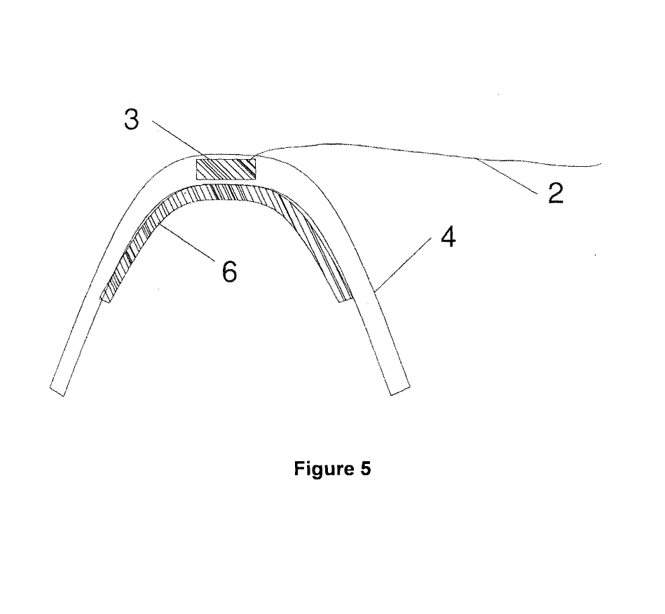

The invention will be shown with FIG. 1, which presents the cross-section of a representative embodiment of the basic design concept of our headband that uses the inductive coil, and illustrates one way the inductive coil can be connected (via cable) to an energy source and the emitter of pulsating frequencies. FIG. 2 shows the same cross-section as FIG. 1, but for a representative embodiment of a wireless transition option for both energy and frequency impulse. FIG. 3 again features a variation of the headband, in this case, with multiple induction coils, capable of influencing various points within the brain. Next we have presented another representation of our innovation as presented in FIG. 4, where the headband has an addition of acoustic earphones, so this variation of the innovation can be used for learning while under the influence of the electromagnetic waves with positively pulsating frequencies. In accordance with some embodiments, we have also created a combination of the headband with our inductive coil and a light source, which complement each other to enhance the acceptance of positive influences from the environment in order to relax, which is presented in FIG. 5.

For better understanding of the above mentioned short description of our innovative idea, presented in the form of the headband—a hoop as the impact product that was created upon design of our innovative idea presented in this patent application and its Figures that we will describe in the following text.

FIG. 1: Depicts a cross-section of the basic concept of a headband that involves an inductive coil with a cable connection of the inductive coil to the source of power and emitter of pulsating frequencies—GSM, in accordance with a representative embodiment.

FIG. 2: Depicts a cross-section of the basic concept of our headband that involves the inductive coil with the wireless connection of the inductive coil to the source of power and emitter of pulsating frequencies—GSM, in accordance with a representative embodiment.

FIG. 3: Depicts a cross-section of the basic concept of our headband that involves multiple (two or more) inductive coils, in accordance with a representative embodiment.

FIG. 4: Depicts a cross-section of the basic concept of our headband that involves the inductive coil in a combination with acoustic earphones, in accordance with a representative embodiment.

FIG. 5: Depicts a cross-section of the basic concept of out headband that involves an inductive coil in a combination with a light source, in accordance with a representative embodiment.

The embodiment presented in FIG. 1 presents a cross-section of the headband 4, in which an inductive coil is built-in 3. In some embodiments, the inductive coil 3 is connected to a GSM device 1 by a cable 2. The GSM device 1 functions as a source of power and a source of emitting the pulsating frequencies. The inductive coil 3 has a copper (Cu), or other suitable winding with the wire thickness of 0.1 mm to 0.8 mm (or any other suitable diameter) winded around the construct of thin iron (Fe) plates (and/or another suitable object) that form an iron core at the end. The copper wire, with both ends joined to the leading cable 2, for which we recommend, in some embodiments, to be 0.8 m long (but can be of any desired length). Through this leading cable 2 the impulses from the source of power and the emitter of pulsating frequencies come from the GSM 1. In accordance with some embodiments, the coil 2 produces the electromagnetic field frequency of GSM 1, with the strength of the field being approximately 0.6 mT. Due to the simplicity of such an inductive coil 3, the functionality of the usage as well as both size and strength of the inductive coil 3 and the shape of the inductive coil 3 can vary. All the mentioned attributes can be adapted to the purpose of usage (the achievement of the desired state) and with it connected changes in the combination of frequencies and their duration.

Next we will present the usability of some embodiments of our innovation. For this purpose we made the variation sample (FIG. 2) of our product. In accordance with some embodiments, the headband 4 for wireless receiving of low frequency impulses with the inductive coil 3 from the cell phone GSM device 1 (or any other suitable device) as the impact product that was created upon our design of our innovative idea shown in this patent application and its Figures that we will describe in the text. This variation of the headband 4 features, apart from the inductive coil 3, the micro-variation of an electronic receiver, that receives radio waves and transforms them into electrical impulses which it passes forth to the winding of the induction coil 3. Some embodiments of the micro-variation of the receiver are not part of the patent application and are available in the free retail network in multiple forms, depending on the strength and construction design. But it is one of components that can determine the price of this variation of our invention. With this variation, the leading cable 2 is omitted.

The continuation of our development work on the headband 4 lead us to the variation with two or more inductive coils 3.1, 3.2 and 3.3 depicted in FIG. 3. Some such variations are based on a desire to have simultaneous influence—the working of the inductive impulses—on more than one state of the consciousness of the brain with a single therapeutic session—meaning the single session of usage of the headband 4 in a combination with GSM and the demand of the desired state, while at the same time either meditate or concentrate.

FIG. 4 presents a multipurpose of usage of a representative embodiment of our innovation, by making a variation where the headband 4 with the inductive coil 3 is outfitted with acoustic earphones 5 making this variation of the headband 4 suitable for studying (among other uses) under the influence of the electromagnetic waves of positive pulsating frequencies. There are education institutions that use such technology to influence the human brain while inducing and expanding the memory capacity of the brain to ease the memorization of more information in a shorter time period.

In accordance with some embodiments, we have also created a combination of the headband 4 and the inductive coil 3 with a light source 6 that complements the reception of positive influences, which is depicted in FIG. 5. It is has been found that various light spectres have positive effects on the state of consciousness of human brains and the entire human organism, thus, in some embodiments, such a method is used with people who wish to relax in a way to use the controls of the software for relaxation, which is part of the complete application installed on GSM 1, depicted in FIGS. 1 and 2. In some embodiments, the application on the GSM 1 is a software that determines the frequency which depends on the selected program or the desired state. In some embodiments, the application allows for 4 programs with various frequencies to help sleep, meditation, quick learning and concentration.

In some embodiments, the frequency of impulses comprises one or more substantially square shapes, so one amplitude raises substantially vertically on the axes until achieving the desired size expressed in Hz and once it reaches the desired amount of Hz it is, in some embodiments, interrupted—shut off in order for the procedure to repeat itself. In some embodiments, this repetition continues for a specific time defined within the software. In some embodiments, the frequency of the impulse is determined and modulated according to the program and when the process is completed it turns itself off and the progressive raise of one amplitude is turned on again. In the same way, in some embodiments, each program of positive influence of the frequency stimulator on our brain is temporally determined and once the process is complete the program installed in the GSM (depicted in FIGS. 1 and 2) shuts down automatically.

In some embodiments, the GSM devices change a low frequency impulse of one or several frequencies simultaneously. This or these frequencies modulate after a certain time so, in some embodiments, we can use the combination of frequencies to achieve various effects that we will describe in the following non-limiting examples.

Variation Case 1:
First frequency is between about 8 and about 12 Hz and defines a light relaxation,
After a time period of about 45 seconds or more the frequency is between 3 and 8 Hz and relaxation enters the state when a human dreams,
After about 45 seconds the frequency lower to about 0.1 to 3 Hz which is configured to cause the person to fall asleep.

Effect: a person relaxes and falls asleep. This is especially important with people that suffer from sleeping disorder and with people that are experiencing a migraine, as a person falls asleep and usually sleeps through the migraine attack.

Variation Case 2:
Frequency coming from the GSM is between about 12 and about 15 Hz and defines the state of enhanced concentration,
After the time-frame of about a few minutes the frequency changes to between about 16 and about 18 Hz—a human has enhanced mental capacities and a strong concentration.

Effect: A person is much better at performing difficult tasks that require strong concentration. The purpose of the testing of proposed development tasks of the frequency stimulator as subject in this patent application was to, as we demonstrated in the two variation cases, to determine the frequency ranges that allow for the optimum effect of our device—the headband and GSM as the source of frequency impulses.

In the chart below we stated some cases of the recommended usage of various frequencies:

| Desired state | Frequency |
|---|---|
| Relaxation | Between about 5 Hz and about 10 HZ for |
| | various stages of relaxation |
| Meditation | Between about 4 Hz and about 7 Hz |
| Sleep | Between about 4 Hz and about 6 Hz as starting |
| | frequency and then a transition to frequencies |
| | between about 1.5 Hz and about 2.5 Hz |
| Creative visualization | Around 6 Hz for a few seconds then to about |
| | 10 Hz |
| Stress reduction | Each frequency after about 11 Hz reduces |
| | stress and calms down |
| Autohypnosis | From around 8 Hz to around 10 Hz |
| Enhanced learning | Between about 7 Hz and about 9 Hz while |
| | listening to a lecture or a recording |

With this we found that the usage of some embodiments of the described systems and methods can enhance the control over our brain and bring it to one of these states, which can be achieved through the usage of our device—the frequency stimulator of low frequencies.

Another indicator of conducted testing was to determine the comparison of various frequencies and their effect on the human head if exposed to a longer time periods of the frequency stimuli. We conducted several tests of various people and, in accordance with some embodiments, optimised the time frame of each dosage of the frequency impulses, which is shown in the chart below.

| | |
|---|---|
| SLEEP with frequencies: | 3.5 Hz 10 Min and 2 Hz 20 Min |
| MEDITATION with frequencies: | 8 Hz 10 Min, 6 Hz 10 Min and |
| | 4 Hz 10 Min |
| ENHANCED LEARNING with | 8 Hz 120 Min |
| frequencies: | |
| CONCENTRATION with | 18 Hz 120 Min |
| frequencies: | |

Third part of showing the technical capability of our frequency stimulator is the comparison of pulsating inductive fields with the existing systems and devices. Today we have various electronic devices at our exposal that produce low frequency pulsating electromagnetic fields. Frequencies are categorized into these basic five groups: Delta, Theta, Alfa, Beta and Gama. Our brains vibrate in one of these frequencies, depending on the state and mood. Brain waves are recorded by an EEG (electroencephalograph). The brainwaves are constantly changing.

| Frequency | Frequency range | State |
|---|---|---|
| Delta | between about 0.1 Hz | Deep sleep, clear dreams, enhanced |
| | and about 0.3 HZ | immune function |
| Theta | between about 3 Hz | Deep relaxation, meditation, enhanced |
| | and about 8 Hz | memory, creativity |
| Alpha | between about 8 Hz | Light relaxation, enhanced learning, |
| | and about 12 Hz | positive thinking |
| Low Beta | between about 12 Hz | Relaxation, enhanced concentration |
| | and about 15 Hz | |
| Midrange | Between about 15 Hz | Enhanced mental capability |
| Beta | and about 18 Hz | |
| High Beta | above about 18 Hz | Complete state of being awake, usual |
| | | working state |

The shown results place some embodiments of our frequency stimulator alongside of other contemporary and extremely expensive devices, we have already mentioned beforehand.

In some cases, the following also have the similar way of functioning:
devices used in linguistic schools with programs for fast learning with influencing the Pituitary gland,
function magnetic resonance (fMRI). A method of fMRI recording gives the best direct illustrations of the activities of a certain brain area during conducting various tasks. We place it among the graphic methods that measure the brain functions in place and time and is noticeable for a good spatial resolution,
Transcranial magnetic stimulation (TMS) is an electro-physiologic method with which we influence the functioning of the brain. Lately it is ever more established in neurosciences, for it is relatively non-invasive and non-disturbing for the subject being examined. With the help of short magnetic pulses, distributed into different rhythmic sequences we stimulate the specific parts of brains, above all the cerebral cortex. In some embodiments, we place a special coil on specific, predetermined areas of the head, allowing the magnetic pulses to enter through the lobe into the brain, causing mild and short electric currents. According to the selected characteristics of the stimulation we can use the TMS to distract the functions of brains and can study what such artificially created distractions mean for the functioning of the specified areas of the brain. We can also use it to stimulate the workings, which is also used for research purposes as a therapy for certain cerebral illnesses.

In some embodiments, the frequency stimulator uses a regular cell phone, which can be in daily use by millions of people around the globe. In some embodiments, the cell phone functions as a generator of electromagnetic impulses. The regular telephones function with frequencies of around 40.000 Hz, but our device would work in the range of 0.1 Hz to 30 Hz.

With this we have justified the usage of our invention in practice, where there is a wide spectre of usage and a multiple use of positive stimulation of various states of human consciousness, as with healing other illnesses that occur as head illness.

The present invention may be embodied in other specific forms without departing from its spirit or essential characteristics. The described embodiments, examples, and illustrations are to be considered in all respects only as illustrative and not restrictive. The scope of the invention is, therefore, indicated by the appended claims rather than by the foregoing description. All changes that come within the meaning and range of equivalency of the claims are to be embraced within their scope. In addition, as the terms on, disposed on, attached to, connected to, coupled to, etc. are used herein, one object (e.g., a material, element, structure, member, etc.) can be on, disposed on, attached to, connected to, or coupled to another object—regardless of whether the one object is directly on, attached, connected, or coupled to the other object, or whether there are one or more intervening objects between the one object and the other object. Also, directions (e.g., front back, on top of, below, above, top, bottom, side, up, down, under, over, upper, lower, lateral, etc.), if provided, are relative and provided solely by way of example and for ease of illustration and discussion and not by way of limitation. Where reference is made to a list of elements (e.g., elements a, b, c), such reference is intended to include any one of the listed elements by itself, any combination of less than all of the listed elements, and/or a combination of all of the listed elements. Furthermore, as used herein, the terms a, an, and one may each be interchangeable with the terms at least one and one or more.

1. A frequency stimulator that is configured to control a dosage of impulses of various low frequencies of electromagnetic waves, the frequency stimulator comprising:
a hoop, which is configured to be placed on a user's head; and
a first induction coil, which is coupled to the hoop,
wherein the first induction coil is configured to be in signal communication with a power source and emitter of pulsating frequencies such that the power source and emitter of pulsating frequencies causes the first induction coil to emit low frequency impulses of electromagnetic waves.

2. The frequency stimulator of claim 1, wherein the first induction coil is in signal communication with the power source and emitter of pulsating frequencies via a leading cable that extends between the hoop and the power source and emitter of pulsating frequencies.

3. The frequency stimulator of claim 1, wherein the first induction coil is in wireless communication with the power source and emitter of pulsating frequencies.

4. The frequency stimulator of claim 1, wherein the power source and emitter of pulsating frequencies comprises a programmable electronic device.

5. The frequency stimulator of claim 1, wherein the power source and emitter of pulsating frequencies is configured to cause the first induction coil to create low frequency impulses of electromagnetic waves with an impulse strength of approximately 0.6 mT and of a substantially rectangular shape between about 0.1 Hz and about 30 Hz in a way that one amplitude raises vertically along an axis until a desired value is reached and then shuts off to repeat production of the low frequency impulses of electromagnetic waves.

6. The frequency stimulator of claim 1, wherein the first induction coil is installed within the hoop.

7. The frequency stimulator of claim 6, further comprising a second induction coil that is coupled to the hoop and is that configured to be in signal communication with the power source and the emitter of pulsating frequencies.

8. The frequency stimulator of claim 1, wherein the hoop further comprises two acoustic sound producing devices that are each configured to be held in proximity to an ear of the user when the hoop is placed on the user's head.

9. The frequency stimulator of claim 1, wherein the hoop further comprises a light source.

10. The frequency stimulator of claim 3, wherein the power source and emitter of pulsating frequencies comprises at least one of a cell phone, a tablet, and a laptop.

11. The frequency stimulator of claim 1, wherein the hoop further comprises an electronic receiver that is configured to receive electronic waves from the power source and emitter of pulsating frequencies and to transform the electronic waves into electrical impulses that are communicated to the first induction coil.

12. A system for producing low frequency electromagnetic waves, the system comprising:
a headband, which is configured to be placed on a user's head;
a first induction coil, which is coupled to the headband; and
a power source and emitter of pulsating frequencies,
wherein the first induction coil is in signal communication with the power source and emitter of pulsating frequencies such that the power source and emitter of pulsating frequencies causes the first induction coil to emit a low frequency impulse of an electromagnetic wave.

13. The system of claim 11, wherein the headband further comprises an electronic receiver that is configured to receive electronic waves from the power source and emitter of pulsating frequencies and to transform the electronic waves into electrical impulses that are communicated to the first induction coil.

14. The system of claim 11, wherein the first induction coil is configured to create a magnetic field with a strength between about 0.03 T and about 0.08 T.

15. The system of claim 11, wherein the headband further comprises an acoustic sound producing device that is configured to be held in proximity to an ear of the user when the headband is placed on the user's head.

16. The system of claim 11, wherein the headband further comprises two acoustic sound producing devices that are configured to be held in proximity to ears of the user when the headband is placed on the user's head.

17. The system of claim 11, wherein the headband further comprises a light source.

18. The system of claim 11, wherein the power source and emitter of pulsating frequencies comprises a programmable electronic device.

19. The system of claim 18, wherein the programmable electronic device comprises at least one of a cell phone and a tablet.

20. The system of claim 13, wherein the first induction coil is in wireless signal communication with the power source and emitter of pulsating frequencies.
